(19) United States
(12) Patent Application Publication
(10) Pub. No.: US 2026/0155237 A1
(43) Pub. Date: Jun. 4, 2026

(54) METHOD FOR PROVIDING MEDICAL IMAGING WORKFLOW SUPPORT DATA

(71) Applicant: Siemens Healthineers AG, Forchheim (DE)

(72) Inventors: Alexander KATZMANN, Fuerth (DE); Oliver TAUBMANN, Weilersbach (DE); Michael SUEHLING, Erlangen (DE)

(73) Assignee: Siemens Healthineers AG, Forchheim (DE)

(21) Appl. No.: 19/402,064

(22) Filed: Nov. 26, 2025

(30) Foreign Application Priority Data
Nov. 29, 2024 (EP) 24216487.9

(51) Int. Cl. G16H 30/20; G06F 40/40; G06N 3/0455; G06T 7/00; G16H 10/60

(52) U.S. Cl. CPC G16H 30/20; G06F 40/40; G06T 7/0012; G16H 10/60; G06N 3/0455; G06T 2207/10081

(57) ABSTRACT

A computer-implemented method for providing medical imaging workflow support data, comprises: receiving natural language data, the natural language data including clinical information; generating structured information by applying a large language model to the natural language data, the structured information including the clinical information in a structured format of at least one input node of an inference graph; calculating the medical imaging workflow support data by applying at least one rule of the inference graph to the structured information; and providing the medical imaging workflow support data.

### CROSS-REFERENCE TO RELATED APPLICATION(S)

The present application claims priority under 35 U.S.C. § 119 to European Patent Application No. 24216487.9, filed Nov. 29, 2024, the entire contents of which is incorporated herein by reference.

### FIELD

In one aspect, one or more example embodiments of the present invention relate to a computer-implemented method for providing medical imaging workflow support data. In other aspects, one or more example embodiments of the present invention relate to a data processing system, a medical imaging system, a computer program product and a computer-readable storage medium.

### BACKGROUND

In clinical IT systems, patient information is typically stored fragmented, widely distributed over various IT systems, and often present in the form of unstructured reports only. As was shown by recent studies, this may result in a significant loss of vital information, directly impacting timely and accurate patient treatment, and therefore poses a significant obstacle toward efficient workflow automation and accurate patient care.

At the same time, scanning complexity is rising, primarily due to technological advancements, a shift from the traditional one-size-fits-all model to a personalized medicine approach, and, not least, increasing legal requirements. This situation is exacerbated by a growing staff shortage in healthcare settings, resulting in a widening gap between workload on the one hand and available personnel on the other.

Under these circumstances, missing information often translates into delayed or disrupted care for a substantial portion of patients, which may significantly increase risks and is compromising outcomes. While standardized vocabularies and protocols (e.g. ICD-10, FHIR, . . . ) exist, their practical application within diverse clinical IT landscapes is far from omnipresent, in particular due to data silos, vendor-specific solutions, and, if using non-standardized formats, the inherent ambiguity of natural language in medical documentation.

Therefore, crucial details are often lost in translation from unstructured reports to structured formats, if at all, impacting downstream processes and increasing the risk of delayed or disrupted treatments. Furthermore, clinicians must manually decipher complex reports, map findings, and adapt protocols, diverting valuable time and effort from patient care. Moreover, with increasing complexity of image acquisition techniques, the process of mapping clinical questions into acquisition protocols, as well as their manual refinement to achieve patient-tailored imaging further increases workload and cognitive burden, and results in a reduced focus on the patient.

Due to the imperfection of the available information, decisions are often intuitive and/or based on substantial experience of the treating personnel. This process may lack transparency, explainability and/or traceability, making it challenging to understand rationale and update workflows in response to evolving clinical practices and technological advancements.

In many cases, scanners typically only receive abstract descriptions of the procedure to be performed via the DICOM Modality Worklist (DMWL), which in many cases is insufficient for advanced workflow automation. For example, on-the-fly adaptation of protocols may be limited to user-provided answers to predefined questions based, for example, on decision trees, and/or a manual setting of parameters and steps to be performed. This process relies fully on the expertise of the scanner-operating technologists and often requires time-consuming additional communication with radiologists and referring physicians in case of missing or unclear information.

Further, general scan and postprocessing Standard Operating Procedures (SOPs) are thus still largely modeled as individual specialized protocols, which may in practice lead to a large and complex set of protocols that can be difficult to maintain and requires expert knowledge to properly set up via the corresponding configuration UIs. This is usually done by lead technologists based on requirements posed by the radiologists. If they need support in this process, both in terms of what the best options are for a particular scanner and how to perform the configuration correctly, they are often limited to browsing manuals or asking application specialists who may not be aware of every relevant aspect due to the abundance of options.

The creation and maintenance of scan protocols at the scanner is a costly and time-consuming process. This is primarily a result of the numerous specialized protocols required to meet clinical demands on the one hand and the increasing capabilities, and thus configuration parameters of the scanners on the other. Different scanners with varying capabilities and software versions further complicate the representation of scan protocols. Moreover, on-site, scan protocols are typically adapted and thus specific to each clinic, making it impossible to provide all relevant configurations ex-factory. Therefore, each site maintains its own protocols, e.g., in a protocol book, and ensures they are accurately reflected on each scanner.

For example, CT scan protocols can be exported and imported from and to a scanner as XML files using a proprietary internal schema. However, this schema is not explicitly defined and is only understood through its implementation. Modifying protocol files requires extensive knowledge and understanding due to their large and complex nature. Furthermore, each new scanner software version may require a separate definition and implementation for transferring earlier protocols or protocol versions. Some complementary aspects, such as the clinical decision trees associated with the protocols, may be defined in separate files with similarly specified formats.

Given an abstract high-level protocol specification for a particular patient, scanner-specific protocols currently would have to be selected and configured separately for each scanner, as acquisition and post-processing parameters differ with every scanner and version. Therefore, handling and maintaining scan protocols is often challenging.

The typical process of creating and maintaining scan protocols is manual and relies heavily on the expertise of protocol administrators. This labor-intensive task requires a thorough understanding of the protocols to ensure their accurate implementation and ongoing maintenance, both in a central document (protocol book) and directly on each of the scanners using a user interface for exam protocol definition. This manual process is costly and requires extensive knowledge of the different scanners, software versions, and protocols used at each site. This becomes even more crucial for photon counting computed tomography, which offer many additional capabilities, such as always-on spectral acquisition and ultra-high resolution reconstructions.

Prior art related to this technical field is disclosed, for example, in EP 4 462 445 A1.

### SUMMARY

An underlying technical problem of one or more example embodiments of the present invention is to provide an alternative and/or an improvement to already known solutions for medical imaging workflow support. At least this problem is solved by the subject matter of the independent claims. The dependent claims are related to further aspects of one or more example embodiments of the present invention.

Independent of the grammatical term usage, individuals with male, female or other gender identities are included within the term.

In one aspect, one or more example embodiments of the present invention relate to a computer-implemented method for providing medical imaging workflow support data, the method comprising:
receiving natural language data, the natural language data comprising clinical information,
generating structured information by applying a large language model to the natural language data, the structured information comprising the clinical information in a structured format of at least one input node of an inference graph,
calculating the medical imaging workflow support data by applying at least one rule of the inference graph to the structured information, and
providing the medical imaging workflow support data.

Using inference graphs, rules can be implemented using an implication-based, easily understandable format, making the whole process traceable, editable, and explainable even for complex rulesets, such as resulting from clinical standard operating procedure (SOP) guideline data. The inference graph may allow radiologists and technologists to modify preferred workflows through natural language interactions utilizing natural language-(NL)-based finding definitions. The inference graph may allow a rule-driven derivation of protocols based on the structured information items.

Thus, a configurable rule set component may be provided, specifically allowing clinicians and technologists to adjust workflows to their unique requirements, complying with the SOPs defined by individual clinics, even when using natural language only. Furthermore, the proposed solution allows removing the dependency of steadily increasing context sizes to ensure constant learning capabilities, and introducing a novel data-driven learning mechanism allowing to derive SOPs automatically by using the available sets of paired image and indication/report data as provided by the clinical RIS.

The clinical information may be, for example, patient-specific clinical information. In particular, the natural language data may comprise the clinical information in an unstructured format. For example, the natural language data may comprise an unstructured report, the unstructured report comprising the clinical information. The clinical information may comprise, for example, information regarding demographic parameters, in particular gender, weight, height and/or age, physiological parameters, previous findings, previous treatments, a clinical history and/or an anamnesis of a patient. The clinical information may comprise, for example, a referral request and/or an indication and/or a reason for a medical imaging examination.

The large language model may be configured to recognize and/or understand natural language and, in particular, individual items such as words in input containing natural language and transfer the items into a text output. The large language model may be trained and/or machine-learned. The large language model may be provided, for example, by holding the large language model available in a suitable storge accessible by a computing unit executing the method and/or by downloading it by said computing unit.

The large language model may be used for transforming unstructured information into the structured format and/or vice versa. Typical large language models have a specific strength in summarizing and presenting information in the way the user prompts the large language model to do. They have an inherent capability of extracting information from text. The large language model may be configured for natural language processing (NLP), in particular for NLP-based extraction of the structured information from the natural language data.

For example, the structured information may comprise a structured report, the structured report comprising the clinical information in the structured format. The structured format may be, for example, a predefined and/or computer-readable format. The natural language data and/or the structured information may be text-based.

In the structured format, the clinical information can be easily extracted and efficiently communicated in a machine-readable format using predefined, automated communication protocols. Notably, by utilizing a large language model capable of language understanding rather than only parsing, the approach circumvents a variety of major issues arising from the use of merely parsing-based approaches, such as the mentioned inversion problem. In particular, the structured information can be easily parsed for downstream applications, including the integration of additional classical algorithms, or prompting the user to provide the yet missing information, if needed. The structured information may comprise a set of structured information items. These could be represented, for example, by codes from commonly used coding systems (ICD-10, LOINC, CPT, . . . ) and/or custom, task-specific representations. In particular, the structured information may comprise codes, e.g., ICD-10 or CPT codes, despite the codes not being present in the natural language data.

The medical imaging workflow support data may be, for example, medical imaging decision support data. The medical imaging workflow support data may be calculated, for example, by applying a function for calculating the medical imaging workflow support data to the structured information. The function for calculating the medical imaging workflow support data may be the at least one rule of the inference graph. The function for calculating the medical imaging workflow support data may be trained, in particular machine-learned. For example, the large language model may comprise the function for calculating the medical imaging workflow support data. As an alternative, the function for calculating the medical imaging workflow support data may be rule-based.

A specific advantage of the proposed method results from its strong comprehensibility. As information is presented in a highly structured format, it is easily possible to spot inconsistent information through a human, and thus to reduce novel sources of error as they might result from ontology-based approaches. Further, the reconfigurability of the large language model allows for a simple adaption in case of updated report templates, novel tasks and diseases, and finally the update of previously created structured reports, as reconfigurations correspond to changes in the query, but not changes in the architecture of the large language model.

The large language model may comprise a transformer network. A transformer network is a neural network architecture generally comprising an encoder, a decoder or both an encoder and decoder. In some instances, the encoders and/or decoders are composed of several corresponding encoding layers and decoding layers, respectively. Within each encoding and decoding layer is an attention mechanism. The attention mechanism, sometimes called self-attention, relates data items (such as words or pixels) within a series of data items to other data items within the series. The self-attention mechanism for instance allows the model to examine a word within a sentence and determine the relative importance another word within that sentence has to the word being examined.

The encoder, in particular, may be configured to transform the input (a medical image or text) into a numerical representation. The numerical representation may comprise a vector per input token (e.g., per word). The encoder may be configured to implement an attention mechanism so that each vector of a token is affected by the other tokens in the input. In particular, the encoder may be configured such that the representations resolve the desired output of the transformer network. The decoder, in particular, may be configured to transform an input into a sequence of output tokens. In particular, the decoder may be configured to implement a masked self-attention mechanism so that each vector of a token is affected only by the other tokens to one side of a sequence. Further, the decoder may be auto-regressive meaning in that intermediate results (such as a previously predicted sequence of tokens) are fed back. According to some examples, the output of the encoder is input into the decoder. Further, the transformer network may comprise a classification module or unit configured to map the output of the encoder or decoder to a set of learned outputs such as the text summary.

Training of a transformer model according to some examples may happen in two stages, a pretraining and a fine-tuning stage. In the pretraining stage, a transformer model may be trained on a large corpus of data to learn the underlying semantics of the problem. Such pre-trained transformer models are available for different languages. For certain applications described herein, the fine-tuning may comprise further training the transformer network with medical texts with expert annotated meanings and/or medical ontologies such as RADLEX and/or SNOMED. With the latter, in particular, the transformer model according to some examples may learn typical relations and synonyms of medical expressions.

The large language model may comprise, for example, a transformer architecture. Transformer networks may use the previously inserted text to find a logical continuation by querying the already provided information, extracting key information, and assigning a value to each. Conducting this process layer by layer, the large language model predicts one token (i.e., word or word part) at a time, with each token being based on the provided information, as well as the world knowledge that is implicitly represented in the network weights.

A query may be received, the query comprising the natural language data and further query data, wherein the structured information is generated by applying the large language model to the natural language data and the further query data. The query may comprise, for example, a user query. The user query may be a free text query the user may input into a user interface, e.g., by typing the user query into an appropriate input filed in the user interface or by voice command. The further query data may comprise template data, the template data being indicative of the structured format. In particular, the large language model may be provided with a dynamically configurable template and queried to transform the natural language data, thereby filling the relevant information into the provided template.

Reference data may be received, the reference data being indicative of rules and/or examples related to the generating the structured information and/or related to the calculating the medical imaging workflow support data, wherein the large language model is tuned based on the reference data, in particular self-tuned and/or fine-tuned based on the reference data.

The large language model may be configured to consider the reference data as an additional source of information, in particular in form of general medical guidelines, site-specific rules and/or preferences, and/or the patient history. The examples may be related to different patients and/or examinations. In particular, the reference data may comprise a corresponding preferred medical imaging decision for different sets of exemplary clinical information. In particular, the further query data may comprise the reference data. In particular, the reference data may be automatically injected as part of the query. Based on the reference data, the large language model may identify patterns and/or reasons why certain recommendations for a new patient and/or exam are desirable, thus allowing, for example, for an on-the-fly learning.

The structured information may comprise a structured information item (SII). The structured information item may comprise the clinical information in the structured format of the at least one input node of the inference graph. The structured information item may be related, for example, to clinical findings and/or the operational coding systems (ICD-10, LOINC, CPT). Structured information items may also be modeled using a description-based natural language variable definition. In an exemplary definition, a structured information item may be specified through natural language only. An exemplary definition would be, e.g., “mention of abnormal Chest XR”, “significant artifacts caused by motion”, or “tall person (>180 cm)”. The definition of a structured information item may further comprise possible target values (e.g. true/false), document selectors, and a potential FHIR link.

In particular, a structured information item may comprise one or more of the following properties.
Name: The name of the structured information item. This property may be particularly used in the Inference Graph described below.
Description: A natural-language based description of the structured information item.
Allowed values/value ranges: A definition of allowed values, either in form of a list (e.g. [True, False]), range (e.g. [1 . . . 10]) or data type (e.g. bool/int/string/. . . ).
Document selector: Used to select document types from which this finding may be extracted. This is particularly important if structured information items should be considered relevant only if stemming from a specific source, such as a confirmed previous examination report, a laboratory result, etc.
Equivalent Code: An equivalent code in a classical coding scheme, e.g. FHIR, used to link the structured information item to an already existing information system, if any.

Structured information items may be interchangeable with other sources of information, such as FHIR information, or any format of clinically approved findings. The equivalent code therefore serves as a mechanism to link description-based items to specific, e.g. FHIR-based, definitions so that already approved findings may supersede NL-based ones, further improving reliability.

According to a further aspect, the medical imaging workflow support data corresponds to a workflow aspect node of the inference graph. According to a further aspect, the inference graph is based on clinical procedure guideline data and/or on sets of paired patient and workflow data. According to a further aspect, reference data is received, wherein the inference graph is generated by applying a retrieval-augmented generation model to the reference data.

An inference graph may correspond to a set of functions and related parameters and/or may correspond to a graphical representation that illustrates the relationships (edges) between different pieces of information (nodes). It is used to model how conclusions (inferences) can be drawn from given data based on logical connections and/or probabilistic reasoning. Inference graphs help in understanding and visualizing the flow of information and the logical steps taken to reach a conclusion, which is particularly useful in complex NLP tasks such as question answering, information extraction, and reasoning.

The inference graph may be, for example, a trained inference

graph model. The inference graph may be, for example, a dynamic inference graph, in particular a dynamically updated inference graph. The clinical procedure guideline data may be, for example, clinical standard operating procedure (SOP) guideline data. The reference data may comprise the clinical procedure guideline data and/or the sets of paired patient and workflow data. The retrieval-augmented generation (RAG) model may first retrieve relevant information from the reference data and/or from the sets of paired patient and workflow data. Then, the retrieval-augmented generation model may use a generative model to generate the inference graph based on the retrieved information. The retrieval-augmented generation model thus combines the strengths of retrieval-based and generation-based models.

Structured information items may be linked in the Inference Graph, in particular to derive a patient-tailored acquisition workflow. The inference graph may comprise a ruleset and/or at least two different types of nodes, denoted, for example, as Information Nodes and Workflow Aspect Nodes. The nodes may correspond to variables.

Information Nodes may be an abstract representation of any kind of information. Information nodes comprise a name, description and—during inference—value. Information Nodes may be, for example, Input Nodes or Intermediate Nodes. In the inference graph, each structured information item is represented as an Input Node. Values of Input Nodes may be populated at the beginning of the inference, while values of Intermediate Nodes may be set through rule application. Information Nodes may not directly correspond to acquisition settings (e.g. keV, mAs, pitch, . . . )

Workflow Aspect Nodes may directly correspond to imaging workflow settings and may represent entities such as scans, reconstructions, or post-scanning algorithms. Notably, a Workflow Aspect Node might cover multiple sub-settings, such as a mAs value during scanning, a specific kernel for reconstructions, or a specifically-chosen parameter for an algorithm. In particular, the workflow aspect node of the inference graph may be a target node of the inference graph. These issues may be resolved by bridging the gap between unstructured clinical narratives and the structured data required for efficient workflow automation. By enabling radiologists and technologists to define structured information items (SIIs) only using natural language and integrating them into a rule-driven system, thereby introducing an LLM-powered paradigm shift.

Natural Language Understanding may be used for Seamless Data Extraction. In particular, large language models (LLMs) may be used to autonomously extract relevant clinical information from unstructured reports, translating it into a standardized, machine-readable format represented as structured information items (SIIs) defined only through natural language. This removes the manual mapping bottleneck and ensures data completeness. Furthermore, rules may be created and managed in an intelligent way. In particular, structured information items, expressed in plain language, may form the foundation for creating inference graphs and adaptable rules governing workflow processes in computed tomography imaging. This empowers clinicians and technologists to modify acquisition workflows through natural language interactions, enhancing flexibility and reducing reliance on static rule sets.

An LLM-powered chatbot may offer real-time support, suggesting optimal scan protocols, explaining rule applications, and proactively querying EHR/EMR systems for missing patient context. This fosters data-driven decisions and minimizes the potential for human error. Leveraging the clinical standard operating procedure (SOP) guidelines used for scan workflow configuration and combined with Retrieval Augmented Generation (RAG), large language models (LLMs) can be leveraged to identify the key decision criteria for the scan protocols used at a clinic. This is particularly true for factory protocols, but also ensures adaptability to site-specific SOPs.

According to a further aspect, a representation of the inference graph and/or a representation of a subset of the inference graph is visualized in a user interface, wherein a user input, in particular a manual user input and/or a natural language user input, is received through the user interface, wherein the inference graph is modified based on the user input. The subset of the inference graph may comprise a subgraph of the inference graph and/or a subset of nodes and/or rules of the inference graph. Based on the generated initial rulesets, further refinement using graph-based editing is possible, tremendously reducing the initial effort for workflow and protocol configuration. The graph may be modified using an intuitive graph editing approach. In general, this process is possible in a manual fashion due to the option of visualizing the graph itself. This provides a highly relevant and intuitive fallback solution, in order to accelerate the process of graph modification

Clinical IT systems may store the conducted scans with their respective protocols together and the clinical data of the patient. Therefore, a de-siloing the data may be applied for deriving inference graphs from paired patient and examination data using LLMs through a data mining approach, automatically extracting patient specifics and subsequently analyzing found patterns, allowing to derive general rules that can be represented using the inference graph concept. Notably, the above-proposed data mining approach can similarly be used to analyze continuously changing patterns, as they are typical during the adoption of novel technologies. Therefore, the proposed method allows to slowly adapt with the users as they get more familiar with the novel technologies, and so to further improve clinical workflows over time in a Continuous Learning approach.

According to a further aspect, additional information is queried by an artificial intelligence-based assistance function, in particular in form of a chatbot, from a medical information system and/or from a user. In particular, the inference graph may be modified based on the additional information and/or the medical imaging workflow support data may be calculated based on the additional information. The graph editing interaction may be automated using the artificial intelligence-based assistance function.

In addition to a scan user interface, which would be enriched to display and/or make accessible the derived workflow adaptations, suggestions as well as their basis in terms of rules and source data, an artificial intelligence-based, in particular LLM-based, assistance function, in particular in form of a chatbot, may be used to guide and support the user of the system at any point of the scan and postprocessing workflow. This is of particular importance and benefit considering that making the ideal choices for each patient during imaging and postprocessing grows more complex due to the increasing wealth of possibilities—this holds true especially since the introduction of photon counting CT with “always on” spectral imaging and the possibility to acquire and reconstruct ultra-high resolution (UHR) images.

The indication for the current scan as well as the available structured and unstructured patient history, i.e. the clinical context information which may serve as input for the rule-based workflow automation described above, may also be available to the artificial intelligence-based assistance function. In addition, it may also have access to the rule-set definitions as well as the ideal workflow derived from it based on the clinical context information.

Expert technical and clinical knowledge about the scanner and its capabilities may be provided as well, both by the vendor (as a default, e.g. scanner manuals, whitepapers, . . . ) or by the user themselves (optionally; custom site-specific know-how and/or SOPs that may not be directly covered or fully explained by the rule-set). This way, the assistance function is well-equipped to reason about and answer any questions regarding the patient, the ideal exam and how to best achieve it using the scanner at hand (“smart user manual”).

The technical mechanisms and/or means with which these documents and related information can be made available to the LLM include direct injection in prompts (system prompt, enriched user prompts) in case the context window and capabilities of the LLM allow for the corresponding amount of text to be processed efficiently and usefully, and/or Retrieval Augmented Generation systems where only relevant parts of the documents are searched and injected dynamically, using, e.g., a vector database that stores pre-computed semantic embeddings of all relevant source documents. The documents may be present in natural (e.g., diagnostic reports) and/or structured language (e.g., derived workflow in a format like JSON or XML) where applicable. LLMs may be fit to flexibly deal with both.

Clinical context information in many cases needs to be retrieved from other systems in the hospital, such as an EHR/EMR (electronic health/medical record) or HIS/RIS/LIS (hospital/radiology/laboratory information system). Integration with these systems on a technical (cf. connectivity) and/or semantic (cf. concept mapping) level allows to use information contained for the purposes described herein. However, the employed LLMs may also assist with the dynamic querying of information. For instance, if a certain piece of information is requested by the user (or identified as a required input of a rule within the given rule set) and is not yet known to be accessible, but may be available within a connected system, the appropriate query (e.g., a FHIR query, i.e. a request for an information resource as defined in the FHIR standard, formulated according to the FHIR communication protocol) may be generated automatically by the LLM and then automatically executed in the background to fetch the corresponding information on demand, if present.

As outlined above, the artificial intelligence-based assistance function may support the user with performing the exam. This may not be limited to answering questions and making suggestions in plain text but also include the possibility to determine and navigate to the source of the provided information and conveniently act on the given suggestions. To this end, the LLM can be instructed to use a prescribed markup language when referring to any input documents or pieces of information as well as when referring to workflow parameters and their values. Specifically, the markup could include a combination of functional descriptors (“jump to document/information”, “show parameter value”, “set parameter value”, “show default/allowed values for parameter”, . . . ) and internal unique identifiers for the documents/information and workflow parameters.

This allows the surrounding system to parse the LLM output for this markup and enable call-up of appropriate functions to provide UI & backend elements and interactions as well as display the markup accordingly in the chat (“Function calling”). Examples could include: Any references to information/documents in the text may be displayed as links that allow the user to hover over it, displaying a pop-over with further information such as the name and timestamp of the source document. Clicking on such a link would switch to a more detailed view of the information in the context of the complete documents with timelines to allow for easy verification, tracing and tracking of information.

Similar links for any mentioned workflow steps would allow, on a click, to switch the view to the part of the scan UI where this parameter is set. In case a specific parameter value is mentioned, clicking the link could immediately set the parameter value accordingly in the backend (potentially guarded by a dialog window to confirm to avoid accidental changes). Using automatic LLM function calling, similar interaction may also be triggered by the LLM directly upon user request. As the LLM has received information about all parameters and potential values, it will be able—given appropriate instructions—to identify if a user request refers to non-existent parameters or disallowed values and respond accordingly. Regardless, the corresponding backend functions may be implemented in a way that invalid requests are ignored even if the LLM were to mistakenly include such a request in its response.

In a similar manner, the artificial intelligence-based

assistance function may also allow the user to easily interact not only with the parameters and their values for the current patient, but also the underlying rule set itself. Note that in practice, this typically targets a more specific user role: The rule set, which may reflect the SOPs of the site, would typically be maintained by the lead technologist based on requirements and decisions by radiologists.

As the rule set is represented internally in a formal

language describing the graph of dependencies between information items and predefined workflow steps (see above), natural language requests for new or updated rules can be translated by the LLM into the appropriate format and the resulting modification applied to the current rule set representation. The specification of the formal language for defining the rules, i.e. what types of rules can be expressed in what manner, may be given to the LLM as part of the prompt to ensure correct results as well as the ability to identify infeasible requests or ones that are ambiguous and need further clarification with the user.

The scope of changes can seamlessly range from individual targeted modifications to step-by-step creation of complete graphs or subgraphs based on iterative interaction and/or existing documents that describe the desired rules in prose or a structured format such as a spreadsheet. In this sense, the transition to LLM-powered “rule mining” as described above is gradual. As direct editing of big graph structures—even if done visually with a suitable GUI-tool—can be cumbersome even for experts and is beyond the typical expertise and usual way of working of technologists, this solution is expected to significantly reduce the entry barriers for use of the overall system and increase acceptance and convenience.

The artificial intelligence-based assistance function may be used for context awareness and/or acquisition-aware retrieval. The artificial intelligence-based assistance function may be able to comment on the inference graph and/or the derivation of the currently configured workflow, in particular in form of an interactive scanning guide.

Here is an example listing of a user/chatbot (“Assistant”) interaction showcasing possibilities for direct interaction with the workflow. Note that the markup is displayed as plain text here for clarity only. In the real user interface such markup may be rendered e.g. in analogy to hyperlinks.
User: Can you outline the most important adaptations you made to the protocol and why you made them?
Assistant: Here are the most important deviations from the defaults of the base protocol <jump_to_param=“BaseProtocol”>CT Routine Chest (I−)</jump_to_param>, which is recommended for <jump_to_information_item=CancerStaging>lung cancer staging</jump_to_information_item>:
Changed <jump_to_param=ScanProtocol:: Scan 1::Pitch>pitch</jump_to_param> from 1 to 1.5 because a faster scan can help avoid <jump_to_information_item=MotionArtifacts>motion artifacts</jump_to_information_item> which were observed in a <jump_to_source_document=Report_2024_04_01>prior examination</jump_to_source_document> in April this year.
Turned on <jump_to_param=Visualization::RibUnfolding>rib unfolding</jump_to_param> to facilitate detection of metastatic lesions as the patient has a <jump_to_information_item=“HistoryOfCancer”>prior history of cancer</jump_to_information_item>.
User: Alright, can you set the supercalifragiliticity factor to 3?
Assistant: I'm sorry, I don't recognize ‘supercalifragiliticity’ as a workflow parameter that I am able to modify.
User: Okay, then please at least set the pitch to 100 so the scan goes super fast!
Assistant: Apologies, but the allowed values for pitch within this protocol are <set_param=ScanProtocol::Scan1::Pitch|0.5>0.5</set_param>, <set_param=ScanProtocol::Scan1::Pitch|1>1</set_param>, <set_param=ScanProtocol::Scan1::Pitch|1.5>1.5</set_param>, and <set_param=ScanProtocol::Scan1::Pitch|2>2</set_param>. Would you like me to change one of these or change the <jump_to_param=BaseProtocol>base protocol</jump_to_param>?
User: Please change it to 2, then.
Assistant: Understood. <set_param=ScanProtocol:: Scan1::Pitch|2>Please click here to confirm the change</set_param>.
User: Also, I need to see the spine too, not just the ribs!
Assistant: Do you wish to generate a <jump_to_param=Visualization::SpineStraightening>straightened visualization of the spine</jump_to_param>?
User: Yes!
Assistant: Of course. Please click here to <set_param=Visualization::SpineStraightening|True>confirm</set_param> or reply with “confirm”.
User: confirm
Assistant automatically calls function associated with above markup.

In a further aspect, one or more example embodiments of the present invention relate to a computer-implemented method for providing medical imaging workflow support data, the method comprising:
receiving reference data,-generating the medical imaging workflow support data by applying a retrieval-augmented generation model to the reference data,
providing the medical imaging workflow support data.

Large language models (LLMs) may be used to create and modify protocol files, in particular in form of XML protocol files, and/or other referenced files, such as the decision tree definitions, based on templates and examples, without the need for a complete definition and specification.

A protocol and example database may be dynamically queried using Retrieval Augmented Generation (RAG) to adapt or synthesize protocols from scratch. Additional documents may be provided, such as, for example, protocol excerpts, scanner manuals, and global or site-specific clinical guidelines that get selected dynamically and optimally with respect to the modifications required.

As LLMs may be highly proficient in working with formal languages and can learn by example without modification of the underlying syntactic structure, by using in-context few-shot learning with example protocol files, they can fill in missing information sensibly based on given instructions and common knowledge derived from their training and example data.

This approach enables the creation and adaptation of scanner-compatible protocol files, i.e. the main protocol XML file as well as any other related files that are required for fully defining protocol, based on a more abstract representation of the desired workflow and scan strategy. Thus, the representation can be kept simple and high-level using formal languages (e.g., JSON), the protocol book (typically digitally available, e.g., as a text or table document), and finally natural language interactions, as was already described with regard to the inference graph.

Using this approach, scanner-specific protocols can be automatically derived by selecting example protocol definitions for the appropriate scanner and/or by providing additional information on the scanner capabilities, such as by including the scanner manual as context. Moreover, the same mechanism can be employed to transform abstract, patient-tailored protocol configurations, as created during exam ordering, protocolling or scheduling, in an EHR and/or a cloud-based environment, into a concrete acquisition protocol for a scanner of choice. This enables a fine-grained scan configuration from a UX-friendly fleet-level viewpoint.

A hybrid approach may be required, in particular if a LLM struggles to reliably perform these tasks for complex protocols given their size and context limitations. In a further aspect, a high-level document pre-segmentation using traditional, domain knowledge-driven methods, may be applied. The overall structure of the scan protocol files and the general semantic hierarchy are typically stable over time and similar for different scanners. The relevant sub-parts are then provided to the LLM for further modification, rendering this approach feasible. Thus, while XML-parsing can be used for meaningful pre-segmentation, the detailed modification and adaption may be handled on a semantical level using the LLM.

The proposed solution may allow to automate this process (automatic protocol maintenance, automatic protocol transfer). Optionally, double-checking the results and making minor targeted corrections could be carried out manually and/or through natural language-based instructions. User interactions on cloud-or EHR-level may initiate the automatic transfer of general or patient-tailored protocols to different scanners or scanners of different versions in an automated fashion. Direct natural-language interactions may result in the creation of, or changes to the scan protocols.

A medical imaging examination workflow step, option and/or parameter may be suggested, selected and/or triggered, in particular automatically suggested, selected and/or triggered, based on the medical imaging workflow support data. The medical imaging workflow support data may be indicative of a value and/or a value change of a scan parameter of a scan protocol for a medical imaging examination of a patient by a medical imaging device. The method for providing medical imaging workflow support data may further comprise:-setting the scan parameter and/or conducting a medical imaging examination based on the scan parameter.

The medical imaging workflow support data may be indicative of a value and/or a value change of a reconstruction parameter of a reconstruction algorithm for reconstructing a medical image based on medical imaging data. The method for providing medical imaging workflow support data may further comprise:-setting the reconstruction parameter and/or reconstructing the medical image based on the medical imaging data. The medical imaging workflow support data may be indicative of a value and/or a value change of an image processing parameter of an image processing algorithm for processing a medical image. The method for providing medical imaging workflow support data may further comprise:-setting the image processing parameter and/or processing the medical image. The image processing algorithm may be configured, for example, for post-processing and/or analysis of the medical image. The post-processing of the medical image may comprise, for example, calculating a representation of an anatomical structure based on the medical image.

The structured information can be used, for example, to enhance and/or automate a scan workflow and/or a post-processing workflow. For this purpose, information from the structured report and the unstructured report as well as the referral request may be automatically structured with respect to the clinical questions relevant to the medical imaging exam to be performed, while including all specific aspects surrounding this concern. Exemplarily, based on the given textual information, the large language model can be used to quickly answer relevant questions such as “May the patient receive contrast agent?”, “Is a high-resolution reconstruction recommended for the given indication”, “In which organs should an automated search for metastases be performed?”, and similar.

The structured information, in particular regarding patient demographics, previous findings or comorbidities, may be used, for example, for selecting and/or triggering the appropriate workflows steps, for choosing appropriate parameters for scanning, reconstruction and post-processing, and as prior information for any machine-learning based image processing algorithm run on the resulting image data set.

In a further aspect, one or more example embodiments of the present invention relate to a data processing system for providing medical imaging workflow support data, the data processing system comprising an interface unit and a computing unit,
wherein the interface unit is configured to receive natural language data, the natural language data comprising clinical information,
wherein the computing unit is configured to generate structured information by applying a large language model to the natural language data, the structured information comprising the clinical information in a structured format of at least one input node of an inference graph,
wherein the computing unit is further configured to calculate the medical imaging workflow support data by applying at least one rule of the inference graph to the structured information, and
wherein the interface unit is further configured to provide the medical imaging workflow support data.

The data processing system may be configured for carrying out the method according to one of the aspects of one or more example embodiments of the present invention. The data processing system can comprise, for example, at least one of a cloud-computing system, a distributed computing system, a computer network, a computer, a tablet computer, a smartphone or the like. The data processing system can comprise hardware and/or software. The hardware can be, for example, a processor system, a memory system and combinations thereof. The hardware can be configurable by the software and/or be operable by the software. Calculations for performing an action of a method may be carried out in the processor.

The computing unit may be realized as a data processing system or as a part of a data processing system. Such a data processing system can, for example, comprise a cloud-computing system, a computer network, a computer, a tablet computer, a smartphone and/or the like. The computing unit can comprise hardware and/or software. The hardware can comprise, for example, one or more processors, one or more memories and combinations thereof. The one or more memories may store instructions for carrying out the method steps according to one or more example embodiments of the present invention. The hardware can be configurable by the software and/or be operable by the software. Generally, all units, sub-units, or modules may at least temporarily be in data exchange with each other, e.g., via a network connection or respective interfaces. Consequently, individual units may be located apart from each other.

The interface unit may comprise an interface for data exchange with a local server or a central web server via internet connection for receiving the medial image data sets. The interface unit may be further adapted to interface with one or more users of the system, e.g., by displaying the result of the processing by the computing unit to the user (e.g., in a graphical user interface) or by allowing the user to adjust parameters for data processing or visualization. In other words, the interface unit may comprise a user interface.

Data, in particular the natural language data, the query, the further query data, the reference data and the examination request data, can be received, in particular received through the interface unit, for example, by receiving a signal that carries the data and/or by reading the data from a computer memory and/or by a manual user input, for example, through a graphical user interface. Data, in particular the medical imaging workflow support data, can be provided, in particular provided through the interface unit, for example, by transmitting a signal that carries the data and/or by writing the data into a computer memory and/or by displaying the data on a display.

Any of the algorithms, functions and/or models mentioned herein can be based on one or more of the following architectures: deep convolutional neural network, deep belief network, random forest, deep residual learning, deep reinforcement learning, recurrent neural network, Siamese network, generative adversarial network or auto-encoder.

One or more example embodiments of the present invention further relate to a medical imaging device comprising the data processing system according to one of the aspects of the one or more example embodiments of the present invention. The medical imaging device may be configured for carrying out the medical imaging examination of the patient based on the scan protocol for the medical imaging examination. The medical imaging device may be, for example, a computed tomography device, a magnetic-resonance imaging device, an ultrasound imaging device, a PET imaging device, a SPECT imaging device, an X-ray imaging device, a cone beam CT device and/or a combination thereof.

In a further aspect, one or more example embodiments of the present invention relate to a computer program product comprising instructions which, when executed by a computer, cause the computer to carry out the method according to one of the aspects of one or more example embodiments of the present invention. In a further aspect, one or more example embodiments of the present invention relate to a computer-readable storage medium comprising instructions which, when executed by a computer, cause the computer to carry out the method according to one of the aspects of one or more example embodiments of the present invention. The realization of one or more example embodiments of the present invention by a non-transitory computer program product and/or a non-transitory computer-readable storage medium has the advantage that already existing computers can be easily adapted by software updates in order to work as proposed by one or more example embodiments of the present invention.

The computer program product can be, for example, a computer program or comprise another element apart from the computer program. This other element can be hardware, for example a memory device, on which the computer program is stored, a hardware key for using the computer program and the like, and/or software, for example, a documentation or a software key for using the computer program. The computer program product may further comprise development material, a runtime system and/or databases or libraries. The computer program product may be distributed among several computer instances. A computer-readable storage medium can be embodied as non-permanent main memory (e.g. random-access memory) or as permanent mass storage (e.g. hard disk, USB stick, SD card, solid state disk).

Utilizing a large language model capable of language understanding, rather than only parsing, poses a significant advantage in comparison to symbolic-ontological methods, that typically struggle with negations, inversions, abbreviations, typos and extra information, either being parsed “hardcoded”, and thus very limited, or without being able to handle this information at all. In contrast, language understanding, information extraction and content summarization are key capabilities of typical large language models, allowing to avoid these issues. Specifically, while negations and inversions are an integral part of natural language and thus abundantly represented, world knowledge as well as explicitly given query information can be used to resolve abbreviations and typos. Extra information can be explicitly queried and summarized, such as in form of easily comprehensible bullet points, which themselves may refer to the original report.

As a result, the use of large language models might revolutionize the way in which structured information is created from unstructured natural language data, being a major step toward digital transformation in clinics, improving throughput, accelerating treatment, serving as a double-check for avoiding treatment errors, and finally reducing the workload for the involved parties. With a specific focus on the scan and post-processing workflow automation, such as addressed by the Tech Co-pilot, the advantages are similarly manifold, with the main aspects including time savings through smart scanners, automatically pre-selecting correct workflows and steps, and triggering only relevant and specifically tailored algorithms in order to create relevant (AI-based) image analysis results, standardization, and increased consistency of exams. In summary, this allows for highly reproducible, guidance-based workflow recommendations, and results in a strong potential for error reduction by serving as an automated “second pair of eyes” that is automatically parsing the available text-based sources for any relevant information.

In the context of the present invention, the expression “based on” can in particular be understood as meaning “using, inter alia”. In particular, wording according to which a first feature is calculated (or generated, determined etc.) based on a second feature does not preclude the possibility of the first feature being calculated (or generated, determined etc.) based on a third feature. Reference is made to the fact that the described methods and the described systems are merely preferred example embodiments of the present invention, and that the present invention can be varied by a person skilled in the art, without departing from the scope of the present invention as it is specified by the claims.

### BRIEF DESCRIPTION OF THE DRAWINGS

Features related to the present invention or to the technical background of the present invention will be illustrated below with reference to the accompanying figures. The illustration in the figures is schematic and highly simplified and not necessarily to scale.

FIG. 1 shows a flow chart for a computer-implemented method for providing medical imaging workflow support data.

FIG. 2 shows a data processing system.

FIG. 3 shows a representation of an inference graph.

FIG. 4 shows a user interface for modifying the inference graph.

FIG. 5 shows a flow chart for a computer-implemented method for protocol modification.

### DETAILED DESCRIPTION

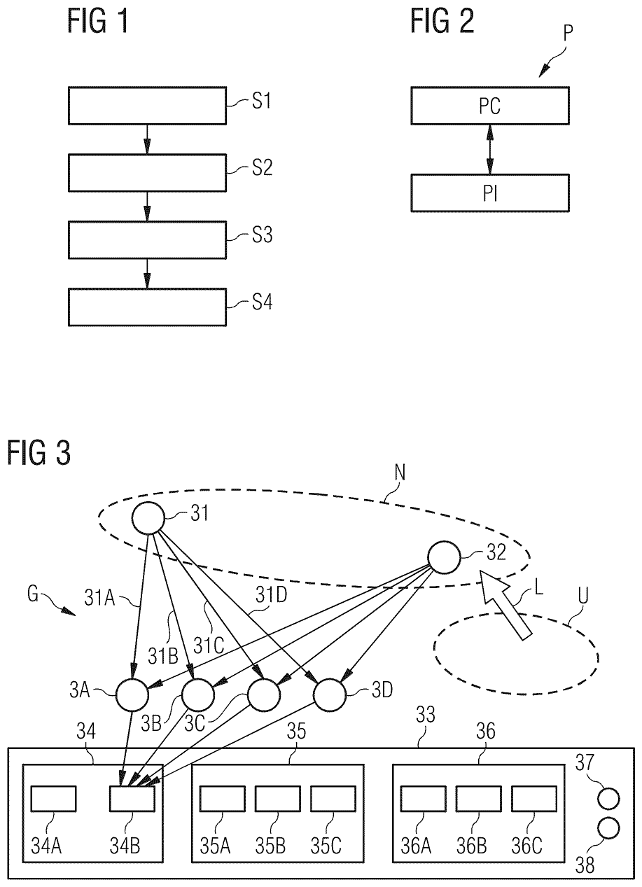

FIG. 1 shows a flow chart for a computer-implemented method for providing medical imaging workflow support data, the method comprising:
receiving S1 natural language data U, the natural language data U comprising clinical information,-generating S2 structured information N by applying a large language model L to the natural language data U, the structured information N comprising the clinical information in a structured format of at least one input node 31, 32 of an inference graph G,
calculating S3 the medical imaging workflow support data by applying at least one rule of the inference graph G to the structured information N, and
providing S4 the medical imaging workflow support data.

FIG. 2 shows the data processing system P for providing medical imaging workflow support data, the data processing system P comprising an interface unit PI and a computing unit PC,
wherein the interface unit PI is configured to receive S1 natural language data U, the natural language data U comprising clinical information,
wherein the computing unit PC is configured to generate S2 structured information N by applying a large language model to the natural language data U, the structured information N comprising the clinical information in a structured format of at least one input node 31, 32 of an inference graph G,
wherein the computing unit PC is further configured to calculate S3 the medical imaging workflow support data by applying at least one rule of the inference graph G to the structured information N, and
wherein the interface unit PI is further configured to provide S4 the medical imaging workflow support data.

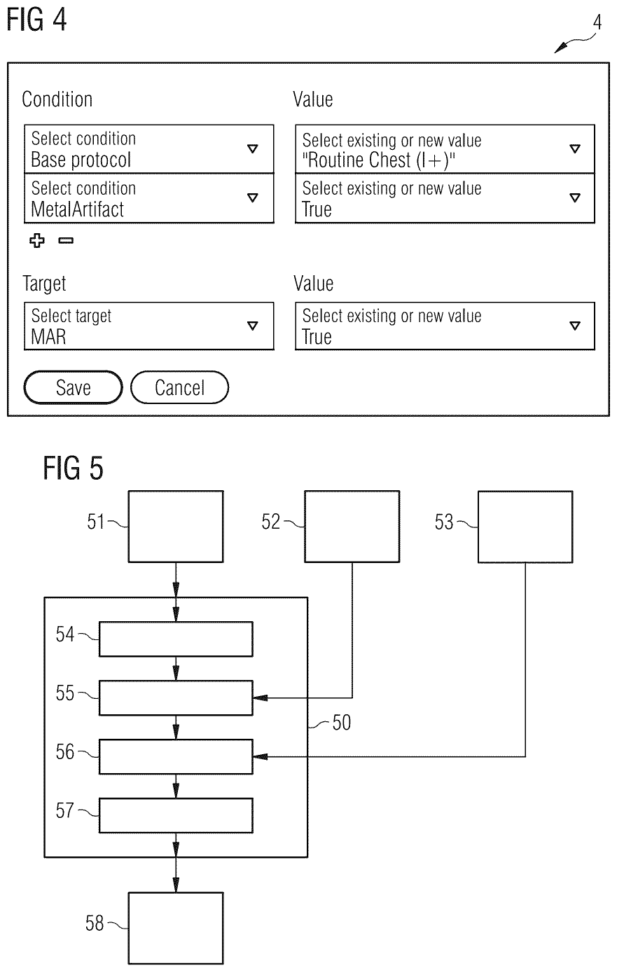

In FIG. 3 and FIG. 4, an exemplary implementation of the manual editing of the inference graph G through the user interface 4 is depicted.

The input node 31 represents a base protocol condition. The input node 32 represents a metal artifact condition. The edges 31A, 31B, 31C and 31D represent Routine Chest (I+), Routine Chest (I−), Nodule Follow-up (I−) and CT PE (I+), respectively, as the potential values of the base protocol. Each of the connections 3A, 3B, 3C and 3D represents a logical conjunction (“AND”). The target nodes within the set 33 are related to reconstruction. The nodes within the subset 34 are related to specific reconstruction algorithms, in particular to iterative reconstruction 34A and to metal artifact reduction 34B, respectively. The nodes within the subset 35 are related to soft thin reconstruction, in particular to a kernel 35A, a slice increment 35B and a slice thickness 35C, respectively. The nodes within the subset 36 are related to dual-energy computed tomography reconstruction, in particular to PBV 36A, VMI 36B and VNC 36C, respectively. Other nodes are related to thick reconstruction 37 and lung thin reconstruction 38.

The medical imaging workflow support data corresponds to a workflow aspect node of the inference graph G. A representation of the inference graph G is visualized in the user interface 4, wherein a user input, in particular a manual user input and/or a natural language user input, is received through the user interface 4, wherein the inference graph G is modified based on the user input. Thus, the inference graph G may be modified using an intuitive graph editing approach based on a visualization of (a subset of) the inference graph G.

FIG. 5 shows a flow chart for a computer-implemented method for protocol modification.

The protocol modification function 50 generates the output protocol file 58 based on the input protocol file 51 and the prompt 53, the prompt 53 comprising a description/request (e.g. “The slice thickness of recon three should be halved.” or the equivalent code) and/or a link to protocol book data. The reference database 52 comprises additional documents such as protocol excerpts, scanner manuals, and/or global and/or site-specific clinical guidelines that get selected dynamically and optimally by the retrieval-augmented generation model with respect to the modifications required.

The protocol modification function 50 comprises the function 54 for pre-segmentation of the input protocol file 51, the function 55 for context-aware retrieval and retrieval-augmented generation, the function 56 for processing the prompt and modifying the protocol file, and the function 57 for a reassembly to obtain the output protocol file.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A computer-implemented method for providing medical imaging workflow support data, the computer-implemented method comprising:
receiving natural language data, the natural language data including clinical information;
generating structured information by applying a large language model to the natural language data, the structured information including the clinical information in a structured format of at least one input node of an inference graph;
calculating the medical imaging workflow support data by applying at least one rule of the inference graph to the structured information; and
providing the medical imaging workflow support data.

2. The computer-implemented method according to claim 1, wherein the medical imaging workflow support data corresponds to a workflow aspect node of the inference graph.

3. The computer-implemented method according to claim 1, wherein the inference graph is based on at least one of clinical procedure guideline data or sets of paired patient and workflow data.

4. The computer-implemented method according to claim 1, further comprising:
receiving reference data; and
generating the inference graph by applying a retrieval-augmented generation model to the reference data.

5. The computer-implemented method according to claim 1, further comprising:
visualizing, on a user interface, at least one of a representation of the inference graph or a representation of a subset of the inference graph;
receiving a user input via the user interface; and
modifying the inference graph based on the user input.

6. The computer-implemented method according to claim 1, further comprising:
querying, by an artificial intelligence-based assistance function, additional information from at least one of a medical information system or a user; and
at least one of
modifying the inference graph based on the additional information, or
calculating the medical imaging workflow support data based on the additional information.

7. A computer-implemented method for providing medical imaging workflow support data, the computer-implemented method comprising:
receiving reference data;
generating the medical imaging workflow support data by applying a retrieval-augmented generation model to the reference data; and
providing the medical imaging workflow support data.

8. The computer-implemented method according to claim 1, wherein at least one of a medical imaging examination workflow step, option or parameter is at least one of suggested, selected or triggered based on the medical imaging workflow support data.

9. The computer-implemented method according to claim 1, wherein the medical imaging workflow support data is indicative of at least one of a value or a value change of a scan parameter of a scan protocol for a medical imaging examination of a patient by a medical imaging device.

10. The computer-implemented method according to claim 1, wherein
the medical imaging workflow support data is indicative of at least one of a value or a value change of a reconstruction parameter of a reconstruction algorithm for reconstructing a medical image based on medical imaging data.

11. The computer-implemented method according to claim 1, wherein
the medical imaging workflow support data is indicative of at least one of a value or a value change of an image processing parameter of an image processing algorithm for processing a medical image.

12. A data processing system for providing medical imaging workflow support data, the data processing system comprising:
an interface unit configured to
receive natural language data, the natural language data including clinical information, and
provide the medical imaging workflow support data; and a computing unit configured to
generate structured information by applying a large language model to the natural language data, the structured information including the clinical information in a structured format of at least one input node of an inference graph, and
calculate the medical imaging workflow support data by applying at least one rule of the inference graph to the structured information.

13. A medical imaging device comprising:
the data processing system of claim 12.

14. A non-transitory computer program product comprising instructions that, when executed by a computer, cause the computer to carry out the computer-implemented method of claim 1.

15. A non-transitory computer-readable storage medium comprising instructions that, when executed by a computer, cause the computer to carry out the computer-implemented method of claim 1.

16. The computer-implemented method according to claim 6, wherein the artificial intelligence-based assistance function is in a form of a chatbot.

17. The computer-implemented method according to claim 2, wherein the inference graph is based on at least one of clinical procedure guideline data or sets of paired patient and workflow data.

18. The computer-implemented method according to claim 17, further comprising:
receiving reference data; and
generating the inference graph by applying a retrieval-augmented generation model to the reference data.

19. The computer-implemented method according to claim 18, further comprising:
visualizing, on a user interface, at least one of a representation of the inference graph or a representation of a subset of the inference graph;
receiving a user input via the user interface; and
modifying the inference graph based on the user input.

20. The computer-implemented method according to claim 5, further comprising:
querying, by an artificial intelligence-based assistance function, additional information from at least one of a medical information system or a user; and
at least one of
modifying the inference graph based on the additional information, or
calculating the medical imaging workflow support data based on the additional information.
